(19) United States
(12) Patent Application Publication
(10) Pub. No.: US 2013/0224819 A1
(43) Pub. Date: Aug. 29, 2013

(54) METHODS AND APPARATUSES FOR PRODUCING BIOGASES

(71) Applicant: Pierre Rivard, Laverlochere (CA)

(72) Inventor: Pierre Rivard, Laverlochere (CA)

(21) Appl. No.: 13/882,890

(22) PCT Filed: Nov. 1, 2011
(86) PCT No.: PCT/CA2011/001215

§ 371 Date: May 1, 2013

Related U.S. Application Data
(60) Provisional application No. 61/410,699, filed on Nov. 5, 2010.

(51) Int. Cl. C12P 5/02

(52) U.S. Cl. CPC C12P 5/023
USPC 435/167; 435/294.1

(57) ABSTRACT

There are provided methods for producing at least one biogas comprising submitting an organic material to an anaerobic digestion process in an apparatus effective for carrying such a process so as to produce the at least one biogas, pressurizing the produced at least one biogas, and using the at least one biogas for conveying the organic material through the apparatus. Apparatuses for producing at least one biogas are also disclosed.

### FIELD OF THE DISCLOSURE

The present disclosure relates to the field of production of biogases. In particular, the present disclosure relates to apparatuses and methods for producing biogases by means of degradation of organic material.

### BACKGROUND OF THE DISCLOSURE

Several systems are known in the art for producing biogases. However, several of them are either very costly to acquire and/or to operate. Moreover, several proposed technologies do not easily allow for producing biogases by using substantially dry organic material having a significant dryness. Often, the starting material is liquefied and then treated (digested), thereby adding an extra step. Moreover, certain apparatuses and methods involve complex mechanical means for producing biogases which complicates the technology and renders exposes the owner to potential high costs in terms of maintenance and in terms of replacement costs in cases of mechanical failure.

### SUMMARY OF THE DISCLOSURE

It would thus be highly desirable to be provided with an apparatus or a method that would at least partially solve one of the problems previously mentioned or that would be an alternative to the existing technologies.

According to one aspect there is provided method for producing at least one biogas comprising submitting an organic material to an anaerobic digestion process in an apparatus effective for carrying such a process so as to produce the at least one biogas, pressurizing the produced at least one biogas, and using the at least one biogas for conveying the organic material through the apparatus.

According to another aspect, there is provided a method for producing at least one biogas comprising :
feeding an apparatus effective for converting organic material into the at least one biogas and conveying the organic material in the apparatus by means of a pressurized gas, wherein the pressurized gas is the produced at least one biogas that has been pressurized.

According to another aspect, there is provided a method for producing at least one biogas comprising
feeding an apparatus effective for converting an organic material into the at least one biogas;

recovering the produced at least one biogas;
pressurizing the at least one biogas; and
using the at least one biogas for conveying the organic material in the apparatus.

According to another aspect, there is provided in a method for producing at least one biogas by using an apparatus effective for carrying out an anaerobic digestion process of an organic material, the improvement wherein the so-produced at least one biogas is pressurized and used for conveying the organic material through the apparatus.

According to another aspect, there is provided in an apparatus for producing at least one biogas for an organic material and in which the apparatus comprises a digester an at least one conduit upstream or downstream of the digester and/or at least one chamber upstream or downstream of the digester, the improvement wherein the so-produced at least one biogas is pressurized and used for conveying the organic material through the apparatus.

According to another aspect, there is provided an apparatus for producing at least one biogas from an organic material, the apparatus comprising :
a reservoir for receiving the organic material;
at least one digester effective for digesting the organic material and converting at least a portion of it into the at least one biogas, the digester being connected to the reservoir by means of a conduit;
means for conveying the organic material from the reservoir to the digester; and
wherein the means for conveying the organic material comprises means for pressurizing the produced biogas and using the pressurized biogas as a carrier for conveying the organic material.

It was found that by using such methods and apparatuses it was possible to avoid several mechanical failures due to high frictions (more particularly in conduits of the apparatus). Moreover, it was found that by using such methods and apparatuses it was possible to treat solid organic material (such as animal manure (e.g. pork, beef, poultry, etc)) that have a high dryness (for example a dryness of about 15 to about 40% or solid organic material from other sources (such as oat, barley or wheat straw), it was also observed that it was also possible to treat other organic material that have a higher dryness (for example organic material having a high fiber content or a high lignin content (such as about 8 to about 32%) and having, for example, a dryness of about 40 to about 90%). Finally, it was observed that solid organic material from other sources (such as kitchen wastes or municipal solid wastes) that have a high dryness (for example a dryness of about 15 to about 90%, can also be treated by such methods and apparatuses.

According to another aspect of the present disclosure, there is provided the use of a moose rumen, a part thereof or an extract thereof as an inoculant in a process for producing at least one biogas.

According to another aspect of the present disclosure, there is provided the use of a moose rumen, a part thereof or an extract thereof for producing at least one biogas.

According to another aspect of the present disclosure, there is provided the use of a moose rumen, a part thereof or an extract thereof in a digester for inoculating an organic material and producing methane.

### BRIEF DESCRIPTION OF THE DRAWINGS

The following drawings represent examples that are presented in a non-limitative manner.

FIG. 1 is a schematic representation of an example of a method and apparatus according to the present disclosure.

### DETAILED DESCRIPTION OF THE DISCLOSURE

The following examples are presented in a non-limitative manner.

The apparatuses and methods described in the present document can be used for production of various biogases such as methane production from the degradation of organic solids. The anaerobic digestion system can include at least one or at least two organic anaerobic treatment units and at least one or at least two biogas container units. The biogas containers can be used to hold compressed biogas. One of the organic anaerobic treatment units can be used for the solid phase treatment; the other units can be used for anaerobic digestion of liquid phase treatment. All units can be joined together for gas, liquid and solid transfer between them. Liquid and solid transfers can be done with at least one compressed biogas. The biogas(es) can be compressed to increase purification efficiency and also for liquid and solid transfer between the different units.

The expression “a part thereof” as used herein and when used in combination with the term “rumen” refers, for example, to a part or a component comprised within a rumen. Such a part or component can be, for example, a microoganism, a bacteria, or an enzyme that is found in such a rumen. Such a part or component can be directly obtained or isolated from the rumen or it can be obtained from a culture prepared using the rumen.

The expression “an extract thereof” as used herein and when used in combination with the term “rumen” refers, for example, to an extract obtain from a rumen. The extract can comprise a liquid phase, a solid phase or a mixture thereof. The extract encompasses, for example, any chemical or combination of chemicals found in a “rumen” or that can be prepared using a chemical or chemicals found in a “rumen”. The extract also encompasses chemical or chemicals that are obtained by preparing derivatives of the compounds found in the “rumen” via chemical reaction(s).

For example, the organic material can have a dryness of about 5 to about 90%, 10 to about 90%, about 10 to about 85%, about 20 to about 85%, about 30 to about 85%, about 40 to about 85% 20 to about 90%, 30 to about 90%, about 10 to about 20%, about 10 to about 25%, about 10 to about 30%, about 15 to about 20%, about 15 to about 25%, or about 15 to about 30%).

For example, the at least one biogas can be methane.

For example, the organic material can be converted into the at least one biogas by reacting it with a bovine rumen, a part thereof or an extract thereof or a moose rumen, a part thereof or an extract thereof.

For example, the organic material can be converted into the at least one biogas by reacting it with a moose rumen, a part thereof or an extract thereof.

For example, the organic material can be reacted with the bovine rumen or the extract thereof or the moose rumen or the extract thereof at a temperature of about −10° C. to about 50° C., about −5° C. to about 40° C., about 0° C. to about 30° C., about 10° C. to about 40° C., about 5° C. to about 35° C., about 10° C. to about 30° C., or about 5° C. to about 25° C.

For example, during the reaction between the organic material and the rumen or extract thereof a liquid phase and a solid phase can be obtained, the phases can be separated and can be each digested in a separate digester so as to produce the at least one biogas.

For example, the at least one biogas can be used for conveying the organic material in the apparatus, and the at least one biogas can be effective for mixing the organic material with the rumen or extract thereof.

For example, the organic material can comprise litter.

For example, the organic material can be chosen from wood particles, straw, hay, manure, sphagnum moss, peat moss, and mixtures thereof.

For example, the organic material during the anaerobic digestion process can be at a pH of about 5 to about 8, about 6 to about 8, about 6.0 to about 7.5, about 6.5 to about 7.5, or about 6.5 to about 7.2.

For example, the method can further comprise passing the at least one biogas through a liquid so as to purify the biogas.

For example, the method can further comprise passing the at least one biogas through a liquid so as to purify the biogas, the biogas can comprise CH₄, CO₂ and H₂S and the liquid comprises water, wherein passing the biogas through the liquid allows for reducing the content of CO₂ and H₂S in the biogas, thereby increasing the concentration of CH₄ in the biogas.

For example, the method can comprise pressurizing the biogas and passing the biogas through the liquid so as to at least partially trap the CO₂ and the H₂S therein, and then depressurizing the liquid thereby releasing the trapped CO₂ and H₂S.

For example, the means for pressurizing the produced at least one biogas and using the pressurized biogas as a carrier gas for conveying the organic material can be a compressor.

For example, at least one of the tank and the at least one digester can comprise a separator effective for separating a liquid phase from a solid phase of the organic material.

For example, the at least one digester can comprise a separator effective for separating a liquid phase from a solid phase of the organic material.

For example, the separator can be a perforated funnel provided with a valve and in which opening of the valve allows for transferring the liquid portion below the separator while maintaining the solid portion above the separator.

For example, the funnel can be a perforated trapezoidal funnel.

For example, the apparatus can comprise the at least one digester that is effective for digesting the organic material and separating the solid phase from the liquid phase and wherein the apparatus can further comprise at least one liquid phase digester in fluid flow communication with the at least one digester, the at least one liquid phase digester being effective for digesting the liquid phase recovered downstream of the separator.

For example, the at least one liquid phase digester can be in fluid flow communication with an inlet of the at least one digester, a conduit being effective for providing a liquid for the at least one liquid digester to the inlet of the at least one digester for at least one of pH regulation and inoculation.

For example, the at least one liquid phase digester can be in fluid flow communication with an inlet of the tank, a conduit being effective for providing a liquid for the at least one liquid digester to the inlet of the tank for at least one of pH regulation and inoculation.

For example, the apparatus can further comprise means for mixing constituents of at least one of the solid phase, the liquid phase, and a mixture thereof.

For example, the means for mixing the constituents of at least one of the solid phase, the liquid phase, and a mixture thereof can be the compressor.

For example, the apparatus can further comprise at least one biogas tank in fluid flow communication with the at least one digester, the biogas tank being effective for storing the produced biogas.

For example, the biogas tank can further comprise a liquid effective for trapping at least a portion of contaminants present in the produced at least one biogas.

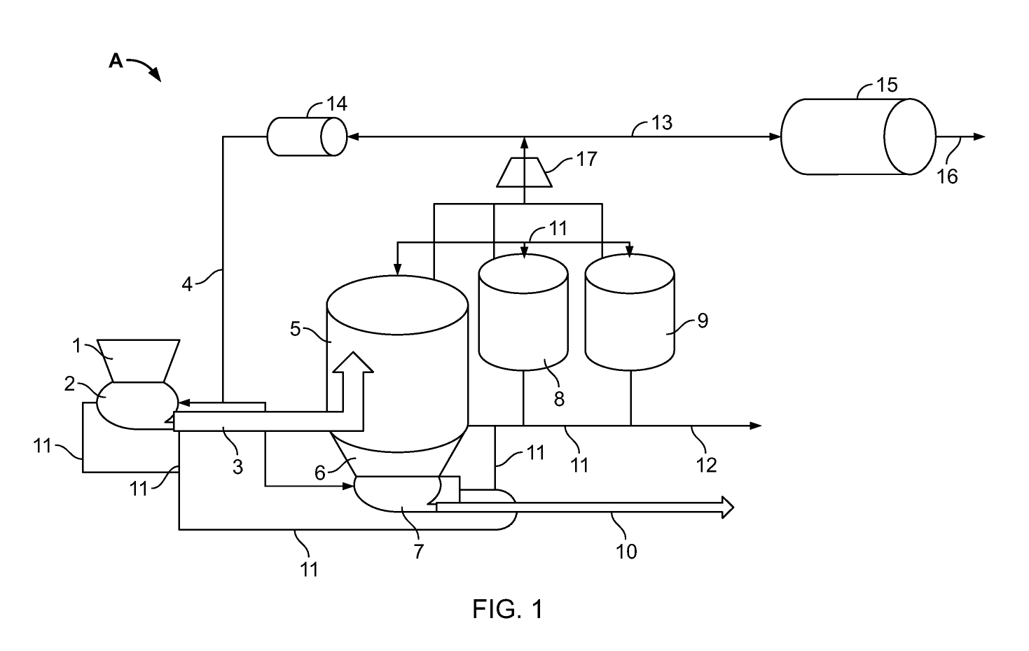

An example of an apparatus and method (schematic representation) is shown in FIG. 1. This drawing is a diagram of flow and storage of the components of the apparatus used for a biogas production system fed with solid wastes.

The various elements in FIG. 1 are as follows :
Inlet of organic solid wastes (1)
First pressure transfer tank (2)
Solid waste transport pipe (3)
Compressed biogas pipe (4)
Solid anaerobic digester (5)
Solid-liquid separator (6)
Second pressure transfer tank (7)
First liquid anaerobic digester (8)
Second liquid anaerobic digester (9)
Solid outflow pipe (10)
Liquid recirculation pipe (11)
Liquid outflow pipe (12)
Biogas distribution pipe (13)
First biogas tank (14)
Second biogas tank (15)
Biogas outflow pipe (16)
Compressor (17)

It has to be noted that for illustration purposes and for simplifying FIG. 1, the valves symbols have been omitted. However, the person skilled in the based on FIG. 1 and on the description of FIG. 1 presented in the present disclosure would be clearly able to understand where the valves can be disposed.

The apparatus (A) of FIG. 1 comprises an inlet of organic solid wastes (1) that can have a trapezoidal shape funnel and it is adapted to receive the solids. The inlet (1) can contain equal or less than the maximum capacity of a first pressure transfer tank (2). Then inlet (1) can be provided with an Auger^{TM} screw. Organic solids can be of many sources: animal manure, crop residues, domestic wastes, sludge and biosolids.

The first pressure transfer tank (2) is adapted to receive the fresh solids. The tank (2) is effective for carrying out inoculation and transporting of the solids towards a solid anaerobic digester (5) by means of a solid waste transport pipe (3). A biogas is introduced in the first pressure tank (2), by means of compressed biogas pipe (4) after introduction of the solids and liquid inoculums in the tank (2). The working pressure varies with the solid content of the mixed substrate. The tank (2) is provided with an opening joined to a trapezoidal funnel. This opening can, for example, be closed with an internal pivot door or a gate valve (not shown) activated by hydraulic or gas pressure force, with an outside mechanism. A biogas inlet is installed at an upper portion of the tank (2), where the tank (2) and the pipe (4) are connected together. Two liquid access pipes (11) are installed near the top and the bottom of the inner tank.

The solid waste transport pipe (3) is a large diameter outflow pipe that is collected at a bottom portion of the tank (2). The pipe (3) is effective for carrying out the material to be digested from the tank (2) to the digester (5). The pipe (3) is equipped with a valve (not shown) near the tank (2) for retention of the solids and liquid present in the pipe from the valve through its exit inside the digester (5). The pipe (3) has a large diameter for easy flowing of the solids.

The biogas produced in the digester (5) eventually passes through a biogas distribution pipe (13) to be compressed by means of a compressor (17) in a first biogas tank (14) and can be transferred under pressure to the tank 1 and into a second pressure transfer tank (7). The biogas can also return to the anaerobic digester (5). The biogas coming from the biogas tank (14) will serve as a compression gas for solids and liquids transfer between the tanks and anaerobic digesters. In fact, it is useful for conveying the material (liquid and solid) between tank (2) and digester (5), for conveying the liquid between the digester (5) and a first liquid anaerobic digester (8) and a second liquid anaerobic digester (9).

The digester (5) has a large capacity and receives the fresh solids from the pipe (3). The solids are pumped to the top of the anaerobic digester (5). The inside bottom of the digester (5) is provided with a solid-liquid separator that can be for example a perforated trapezoidal funnel (6). The separator (6) allows for transferring the solids at the center of it by a large diameter pipe (not shown). This large pipe will be equipped with a valve (not shown). The separator (6) is effective for keeping the solid on the upper side and the liquid on the lower side. The separator (6) (for example a trapezoidal solids funnel) is installed inside the anaerobic digester (5) and is designed to receive the entire volume of solids contained in the digester. The separator (6) can be partly perforated for liquid and small diameter solids flowing through. The liquid can be level controlled. The liquid is then transferred to the tanks (2) and (7) and the liquid anaerobic digesters (8) and (9) by means of the liquid recirculation pipe (11).

The biogas produced from the digesters (5), (8) and (9) is then transferred and compressed to the biogas tank (14) by means of a biogas distribution pipe (13). The biogas tanks (14) and (15) may optionally contain liquids for gas purification.

The pressure transfer tank (7) is similar to the pressure transfer tank (2). The tank (7) will receive the solids digested and will serve to evacuate these solids. The pressure transfer tank (7), like the tank (2), is provided with gas, liquid and solid outlets and inlets.

The digester (8) is adapted to receive the liquids and soluble organics transferred from the digester (5) by means of the pipe (11). This digester (8) is provided with gas outlet, a liquid outflow pipe (12) and inflow pipes. All the pipes can be passing through a bottom portion of the digester (8). The biogas pipe (13) is inside the digester (8) and passes through the liquid and reach the top of the inside digester over the maximum level of liquid inside the digester to capture the biogas produced. The liquid anaerobic digester (9) is identical to the liquid anaerobic digester (8).

A solid outflow pipe (10) is effective for taking the solids out of the tank (7). The pipe (10) is equipped with a valve (not shown) near the tank (7) for retention of the solids and liquid present in the pipe from the valve through its exit. This pipe has a large diameter for easy flowing of the solids.

The pipe (11) allows for the bi-flow direction of the liquids. The liquids will be transferred for anaerobic stabilisation and inoculation in different part of the system as explained before.

The liquid outflow pipe (12) is effective for evacuating the overflow of liquid produced by the system. The liquid outflow will be pumped over the solids evacuated on a concrete slab (not shown), transported for fertilisation purpose or stored in a storage tank facility.

The biogas distribution pipe (13) is effective for distributing of the biogas between the biogas tanks (14) and (15) and the anaerobic digesters (5), (8), and (9). Most of the pipes are bi-direction to facilitate the transfer of solids and liquid between the tanks and the digesters. The tank (14) is adapted to receive the biogas compressed at a range of 5 to 60 psig pressure. The biogas stored in this tank can be or not be treated and/or purified. For example, it can be purified by passing through a liquid such as water under high pressure. Such a purification step allows for solubilizing CO₂ and H₂S in water, thereby increasing the concentration of methane in the biogas. The tank (15) can be adapted to receive the biogas compressed from the tank (14) at a range of 60 to 3600 psig pressure depending on the use of the biogas produced. The biogas stored in this tank can be treated and/or purified depending on the use of the biogas produced. This can be done as detailed above concerning the tank (14).

A biogas outflow pipe (16) can be used for evacuating the biogas produced from the apparatus (A). This outflow will vary depending on the utilisation of the biogas produced.

The organic wastes and solids treated in the apparatus (A) can have variable solid contents (dryness), as previously discussed. The organic wastes are introduced in the tank (2) provided with a trapezoidal shape funnel. The tank (2) can have 9 states of operation: stationary state, entry of solids, preparation to transfer solids, stationary state, entry of compressed biogas, transfer of solids, stop of solids movement, biogas transfer, entry of liquid and stationary state. Pipes for biogas (4), liquid (11) and solids (3) are connected to the tank (2). Before the entry of solids in the tank (2), a portion of the space in the tank (2) is occupied by liquids from the anaerobic digester and the opening for solids is closed. Cow or moose rumen can serve as inoculums. The rumen can be used in its entirety. The rumen can be undamaged. For example, large contact with open air can be avoided or minimised. For example, the rumen can be introduced at the beginning of a sequence of waste transport to the anaerobic digester. The rumen can be placed at the starting period of the digester operation. For example, at the starting period, the weight of rumen can represent at least about 5, 6, 7, 8 or 10% of the material entered for a period of four weeks but not more than 50%. After the starting period, rumen can be added periodically with the wastes entered in the system. When introducing the solids, the same quantity of liquid is transferred by gravity from the tank (2) to tank (7) by opening of the liquid valves (not shown). When all the solids are in place in the tank (2), and liquid is levelled to the opening valve (air present in the tank will be minimum), this one closes. In the next step, the liquid valves are closed and the biogas valve is open, then biogas begins to be introduced in the tank (2) until the pressure reaches a certain pressure. After that pressure is reached, the valve (not shown) of the solid wastes transport pipe (3) is opened and pressure of biogas will be increased until all the solids have passed the valve (not shown) of the solid wastes transport pipe. Then all the valves are closed. The liquid valve (not shown) from the tank (2) is then open to let the biogas be transferred to the tank (7). The valve inlet (not shown) of solids for the tank (7) is opened to solids from the solid anaerobic digester (5) and closed after this operation. When the tank (7) is full of solids and liquid all the valves are closed and a minimum space for gas is present in the tank. Compressed air or waste biogas from the purification process is entered into the tank (7) and pressure is increased to a certain level. Then the valve (not shown) of the solid outflow pipe (10) is open and solids and liquid are evacuated to the solid outflow pipe valve (not shown). Then, the valve is closed. The biogas is transferred to the bottom of the solid anaerobic digester (5) for mixing through the mass of solids. If compressed air is introduced, the air will be evacuated from the system by the introduction of liquid, solid and biogas when the procedure restarts as explained earlier. Then, liquid is re-introduced in the tank (2) and the biogas pressure is lowered until atmospheric pressure by transferring biogas from the tank (2) and tank (7) to the solids anaerobic digester (5).

Liquid anaerobic digesters (8) and (9) are kept at low pressure (for example less than 5 psig). When pressure in the solid anaerobic digester (5) reaches 2 psig, a biogas valve (not shown) on the recirculation pipe (11) opens and transfers the excess pressure of biogas through the liquid anaerobic digesters (8) and (9) alternating for mixing.

Liquids are regularly transferred from liquid anaerobic digesters (8) and (9) and the solid anaerobic digester (5) for pH regulation and inoculation. Levels are controlled and excess of liquids from the solids anaerobic digester is transferred to the liquids anaerobic digester 1 and 2 alternating.

In the methods and apparatuses of the present disclosure, it was found that several types of inoculants can be used for the digestion. For example, bovine or moose rumen, a part thereof or an extract thereof can be used. Tests have been made by the applicant in order to determine the efficiency of such inoculants.

For example, the pH of the organic material during the digestion can be of about 5 to about 8, about 6 to about 8, about 6.0 to about 7.5, about 6.5 to about 7.5, or about 6.5 to about 7.2.

For example, it was found that moose rumen has a productivity that is superior to bovine rumen by more than about 60% and more particularly for example by more than about 64%. Such an increased productivity was observed for up to 120 days. It was also found that the use of moose rumen (as compared to bovine rumen) has allowed for reducing the Chemicals Oxygen Demand (COD) by about 30 to about 40% over a period of about 175 days.

It was also found that anaerobic digestion allowed for reducing the amount of fecal streptococci in bovine manure by about 99% or even more than 99%.

It was also noted when using moose rumen as inoculant it was possible to use an organic material that has a very high dryness. For example, it was observed by the Applicant that the methods and apparatuses of the present disclosure were efficient to digest organic material having dryness as high as 85%. It was thus demonstrated that the methods and apparatuses of the present disclosure can be useful for treating organic material having a high dryness. It was also observed that the methods and apparatuses of the present disclosure can be effective for treating organic material having high lignin content.

Several tests have also been made in order to compare the productivity of the methods and apparatuses of the disclosure when using two types of digesters i.e. a digester for solids and a digester for liquids as opposed to using a single digester. It was found that by using two types of digesters (or separating a solid phase from a liquid phase obtained after a predetermined period of time in the digester) that an increased productivity of the biogas (for example methane) of about 27% was observed over a period of time of 60 days. An increased productivity of about 25% was observed over a period of time of 120 days.

The biogases produced by means of the methods and apparatuses of the present disclosure had a methane content of about 30 to about 70%, a CO₂ content of about 30 to about 70% and a content of H₂S or about 0 to about 0.3%. Such variations occurred between the period of starting the digesters and the time at which a continuous production is reached.

The person skilled in the art would understand that the various properties or features presented in a given embodiment can be added and/or used, when applicable, to any other embodiment covered by the general scope of the present disclosure.

The present disclosure has been described with regard to specific examples. The description was intended to help the understanding of the disclosure, rather than to limit its scope. It will be apparent to one skilled in the art that various modifications can be made to the disclosure without departing from the scope of the disclosure as described herein, and such modifications are intended to be covered by the present document.

1. A method for producing at least one biogas comprising submitting an organic material to an anaerobic digestion process in an apparatus effective for carrying such a process so as to produce said at least one biogas, pressurizing said produced at least one biogas, and using said at least one biogas for conveying said organic material through said apparatus.

2. A method for producing at least one biogas comprising
feeding an apparatus effective for converting an organic material into said at least one biogas;
recovering said produced at least one biogas;
pressurizing said at least one biogas; and
using said at least one biogas for conveying said organic material in said apparatus.

3. The method of claim 1 or 2, wherein said at least one biogas is methane.

4. The method of claim 1 or 2, wherein said organic material is converted into said at least one biogas by reacting it with a bovine rumen, a part thereof or an extract thereof or a moose rumen, a part thereof or an extract thereof.

5. The method of any one of claims 1 to 4, wherein said organic material is converted into said at least one biogas by reacting it with a moose rumen or an extract thereof.

6. The method of claim 4 or 5, wherein said organic material is reacted with said bovine rumen or said extract thereof or said moose rumen or said extract thereof at a temperature of about −10° C. to about 50° C.

7. The method of claim 4 or 5, wherein said organic material is reacted with said bovine rumen or said extract thereof or said moose rumen or said extract thereof at a temperature of about −5° C. to about 40° C.

8. The method of claim 4 or 5, wherein said organic material is reacted with said bovine rumen or said extract thereof or said moose rumen or said extract thereof at a temperature of about 0° C. to about 30° C.

9. The method of claim 4 or 5, wherein said organic material is reacted with said bovine rumen or said extract thereof or said moose rumen or said extract thereof at a temperature of about 10° C. to about 40° C.

10. The method of claim 4 or 5, wherein said organic material is reacted with said bovine rumen or said extract thereof or said moose rumen or said extract thereof at a temperature of about 5° C. to about 35° C.

11. The method of claim 4 or 5, wherein said organic material is reacted with said bovine rumen or said extract thereof or said moose rumen or said extract thereof at a temperature of about 10° C. to about 30° C.

12. The method of claim 4 or 5, wherein said organic material is reacted with said bovine rumen or said extract thereof or said moose rumen or said extract thereof at a temperature of about 5° C. to about 25° C.

13. The method of any one of claims 4 to 12, wherein during reaction between said organic material and said rumen or extract thereof a liquid phase and a solid phase is obtained, said phases are separated and each digested in a separate digester so as to produce said at least one biogas.

14. The method of any one of claims 4 to 13, wherein when said at least one biogas is used for conveying said organic material in said apparatus, said at least one biogas is effective for mixing said organic material with said rumen or extract thereof.

15. The method of any one of claims 4 to 14, wherein said organic material comprises litter.

16. The method of any one of claims 4 to 14, wherein said organic material is chosen from wood particles, straw, hay, manure, sphagnum moss, peat moss, and mixtures thereof.

17. The method of any one of claims 4 to 16, wherein said organic material during said anaerobic digestion process is at a pH of about 6 to about 8.

18. The method of any one of claims 4 to 16, wherein said organic material during said anaerobic digestion process is at a pH of about 6.0 to about 7.5.

19. The method of any one of claims 4 to 16, wherein said organic material during said anaerobic digestion process is at a pH of about 6.5 to about 7.5.

20. The method of any one of claims 4 to 16, wherein said organic material during said anaerobic digestion process is at a pH of about 6.5 to about 7.2.

21. The method of any one of claims 1 to 20, wherein said method further comprises passing said at least one biogas through a liquid so as to purify said biogas.

22. The method of any one of claims 1 to 20, said method further comprises passing said at least one biogas through a liquid so as to purify said biogas, said biogas comprises CH₄, CO₂ and H₂S and said liquid comprises water, wherein passing said biogas through said liquid allows for reducing the content of CO₂ and H₂S in said biogas, thereby increasing the concentration of CH₄ in said biogas.

23. The method of claim 22, wherein said method comprises pressurizing said biogas and passing said biogas through said liquid so as to at least partially trap said CO₂ and said H₂S therein, and then depressurizing said liquid thereby releasing said trapped CO₂ and H₂S.

24. Use of a moose rumen or an extract thereof in a digester for inoculating an organic material and producing methane.

25. The use of claim 24, wherein said organic material has a dryness of about 10 to about 85%.

26. The use of claim 24, wherein said organic material has a dryness of about 20 to about 85%.

27. The use of claim 24, wherein said organic material has a dryness of about 30 to about 85%.

28. The use of claim 24, wherein said organic material has a dryness of about 40 to about 85%.

29. Use of a moose rumen or an extract thereof in a digester as an inoculant in a process for producing at least one biogas.

30. Use of a moose rumen or an extract thereof for producing at least one biogas.

31. In a method for producing at least one biogas by using an apparatus effective for carrying out an anaerobic digestion process of an organic material, the improvement wherein the so-produced at least one biogas is pressurized and used for conveying said organic material through said apparatus.

32. In an apparatus for producing at least one biogas for an organic material and in which the apparatus comprises a digester an at least one conduit upstream or downstream of said digester and/or at least one chamber upstream or downstream of said digester, the improvement wherein the so-produced at least one biogas is pressurized and used for conveying said organic material through said apparatus.

33. An apparatus for producing at least one biogas from an organic material, said apparatus comprising :
a tank for receiving said organic material;
at least one digester effective for digesting said organic material and converting at least a portion of it into said at least one biogas, said digester being connected to said tank by means of a conduit;
means for conveying said organic material from said reservoir to said digester; and
wherein said means for conveying said organic material comprises means for pressurizing said produced at least one biogas and using said pressurized at least one biogas as a carrier gas for conveying said organic material.

34. The apparatus of claim 33, wherein said means for pressurizing said produced at least one biogas and using said pressurized biogas as a carrier gas for conveying said organic material is a compressor.

35. The apparatus of claim 33 or 34, wherein said tank comprises a separator effective for separating a liquid phase from a solid phase of said organic material.

36. The apparatus of claim 33, 34 or 35, wherein said at least one digester comprises a separator effective for separating a liquid phase from a solid phase of said organic material.

37. The apparatus of claim 36, wherein said separator is a perforated funnel provided with a valve and in which opening of said valve allows for transferring said liquid portion below said separator while maintaining said solid portion above said separator.

38. The apparatus of claim 37, wherein said funnel is a perforated trapezoidal funnel.

39. The apparatus of any one of claims 36 to 38, wherein said apparatus comprises said at least one digester that is effective for digesting said organic material and separating said solid phase from said liquid phase and wherein said apparatus further comprises at least one liquid phase digester in fluid flow communication with said at least one digester, said at least one liquid phase digester being effective for digesting said liquid phase recovered downstream of said separator.

40. The apparatus of claim 39, wherein said at least one liquid phase digester is in fluid flow communication with an inlet of said at least one digester, a conduit being effective for providing a liquid for said at least one liquid digester to said inlet of said at least one digester for at least one of pH regulation and inoculation.

41. The apparatus of claim 39 or 40, wherein said at least one liquid phase digester is in fluid flow communication with an inlet of said tank, a conduit being effective for providing a liquid for said at least one liquid digester to said inlet of said tank for at least one of pH regulation and inoculation.

42. The apparatus of any one of claims 35 to 41, wherein said apparatus further comprises means for mixing constituents of at least one of said solid phase, said liquid phase, and a mixture thereof.

43. The apparatus of claim 42, wherein said means for mixing said constituents of at least one of said solid phase, said liquid phase, and a mixture thereof is said compressor.

44. The apparatus of any one of claims 33 to 43, wherein said apparatus further comprises at least one biogas tank in fluid flow communication with said at least one digester, said biogas tank being effective for storing said produced biogas.

45. The apparatus of claim 44, wherein said biogas tank further comprises a liquid effective for trapping at least a portion of contaminants present in said produced at least one biogas.
